(19) United States
(12) Patent Application Publication
(10) Pub. No.: US 2026/0079157 A1
(43) Pub. Date: Mar. 19, 2026

(54) NOVEL POINT OF CARE SYSTEM AND METHOD USED FOR THE SAME

(71) Applicant: tst biomedical electrical Co., Ltd., Taoyuan (TW)

(72) Inventors: Chia-Hsuan Cheng, Taoyuan (TW); Szu-Heng Liu, Taoyuan (TW); Yu-Tung Huang, Taoyuan (TW)

(21) Appl. No.: 18/888,825

(22) Filed: Sep. 18, 2024

(51) Int. Cl. G01N 33/543; G01N 29/02; G01N 29/24; G01N 33/68; G16B 40/00; G16H 10/40; G16H 15/00

(52) U.S. Cl. CPC G01N 33/54373; G01N 29/022; G01N 29/2462; G01N 33/6893; G16B 40/00; G16H 10/40; G16H 15/00; G01N 2291/02466; G01N 2800/32; G01N 2800/347

(57) ABSTRACT

The invention discloses a point of care system. The point of care system includes a multichannel bio-reaction device, a surface acoustic wave (SAW) signal reader and a communication module. The multichannel bio-reaction device is configured to receive an analyte collected from a user and generate a plurality of SAW signals each of which corresponds to at least one of a set of target proteomic markers in the analyte. The SAW signal reader is configured to receive the SAW signals and produce a plurality of time-stamped data corresponding to the SAW signals. The communication module configured to receive the plurality of time-stamped data and transmit the received plurality of time-stamped data to a processor, wherein the processor is configured to run a report-generating process for generating a report including an analysis to the set of target proteomic markers.

### FIELD OF THE INVENTION

The present invention is related to a point of care system, in particular a point of care system including a multichannel bio-reaction device, a surface acoustic wave (SAW) signal reader and a communication module.

### BACKGROUND OF THE INVENTION

Chronic diseases such as diabetes or chronic cardiovascular diseases are more common than ever in the world due to the life style of people living in urban areas. Because of this, certain physiological parameters of those patients with chronic diseases need to be routinely monitored to effectively control their condition so as to avoid deterioration and provide timely treatment.

FIG. 1 shows a conventional medical system for monitoring the patients with chronic diseases. In general, some elderly people might suffer more than one type of chronic disease. On the other hand, there are more than one indicator or marker to be monitored during a health exam, and those indicators or markers can be cross-relevant to several disease. To make it simple, we briefly introduce a typical flow of the conventional medical system for a patient with diabetes.

To begin with the process as illustrated in FIG. 1, a blood sample is to be collected from the body of the patient (Step 11). The patient may either stay home with the help of visiting medical professionals or go to the hospital for a blood test. The blood analyte collected from the patient can be forwarded to a lab for analysis by machine (Step 12).

Depending on the type of markers or indicators to be verified, it could take a period of several hours to several days to report the test result (Step 13). The verified values of some markers or indicators are reviewed by a doctor, along with other medical information related to the patient such as the blood sugar value in recent weeks/months (Step 14). Then, based on the doctor's review, it may be concluded that the patient's condition can be one of a disease (diabetes) (15A), a suspected illness (15B) and a health (15C) conditions.

If the patient is considered as healthy or merely having a suspected illness, monitoring at a regular basis will be required (Step 16). If the disease condition is determined, the doctor may offer a treatment (Step 17) to the patient, and request the patient to take medicine (Step 18) and return to the monitoring process (Step 19) to assure his/her improvement back to health.

Point of care systems, for example the home care or community care functions through the aid of timely communication with remote medical centers, provide good solutions to keep the patients from visiting the hospitals too frequently so as to preserve the precious medical resources for urgent cases. However, only simple physiological indicators such as blood pressure or blood sugar can be detected by the regular home-care devices. Most of those valuable markers or indicators cannot be detected by the home-care devices, and thus there is limited medical information relevant to the patients under the point of care system that can be obtained on a daily basis under the regular point of care management.

According to the abovementioned conventional medical system for monitoring the patients with chronic diseases, it takes time to have the test report based on the analyte collected from the patient due to the limitation of lab resource in the medical center or hospitals, which delays the timing for the treatment of chronic diseases a great deal. In addition, some markers implying middle-term or long-term effect may not be included in each of blood test.

U.S. Pat. No. 10,136,859 provided a method for monitoring some patients suffering obstructive pulmonary disease with a plurality of sensors worn by the patients. Those indicators such as heart rate or blood pressure could relate to lung disease but have limited significance when determining diabetes or chronic cardiovascular diseases.

U.S. Pat. No. 10,921,259 disclosed a hand-held device for detecting physiological indicators from an analyte such as a blood sample. The technology employed by the hand-held device makes use of color detection to the analyte after a reaction has been applied. This method can be good for determining the existence or concentration of some small-molecular indicators such as sugar, but it will be hard to find any proteomic markers (except the HbA1C) which are more significant when determining the health condition of a patient with diabetes or chronic cardiovascular diseases.

Therefore, how to avoid the shortcomings of the above-mentioned medical care system is a technical problem that needs to be resolved.

### SUMMARY OF THE INVENTION

To overcome problems in the prior art, the present invention provides a point of care system employing advanced tools for reading physiological data and making use of the data for more efficient medical care.

According to one aspect of the present invention, there is a point of care system including a multichannel bio-reaction device, a surface acoustic wave (SAW) signal reader and a communication module. The multichannel bio-reaction device is configured to receive an analyte collected from a user and generate a plurality of SAW signals each of which corresponds to at least one of a set of target proteomic markers in the analyte. The SAW signal reader is configured to receive the SAW signals and produce a plurality of time-stamped data corresponding to the SAW signals. The communication module configured to receive the plurality of time-stamped data and transmit the received plurality of time-stamped data to a processor, wherein the processor is configured to run a report-generating process for generating a report including an analysis of the set of target proteomic markers.

According to another aspect of the present invention, there is a method of providing a report for a user adopting a point of care system including a multichannel bio-reaction device, a surface acoustic wave (SAW) reader, a user device, a memory device and a report-generating process running on a processor. The method includes the steps of: the multichannel bio-reaction device receiving an analyte collected from the user, and generating SAW signals based on a set of target proteomic markers in the analyte; the SAW reader receiving the SAW signals, and producing a plurality of time-stamped data corresponding to the SAW signals; the user device receiving the plurality of time-sampled data, and transmitting the received data to the processor; and the report-generating process generating a report including an analysis to the set of target proteomic markers related to the user.

The point of care system can be employed to provide timely analysis reports, which are useful for medical treatment, and the multichannel bio-reaction device and the SAW signal reader can be manufactured to fit the needs of the market. Therefore, the present invention has industrial utility.

### BRIEF DESCRIPTION OF THE DRAWINGS

The objectives and advantages of the present invention will become more readily apparent to those ordinarily skilled in the art after reviewing the following detailed descriptions and accompanying drawings.

FIG. 1 is a schematic diagram showing a conventional medical system for monitoring the patients with chronic diseases known to the art;

FIG. 2 is a schematic diagram showing an analyte collected from a user that may include a set of target proteomic markers;

FIG. 3 is a schematic diagram showing a point of care system according to one embodiment of the present invention;

FIG. 4 is a schematic chart showing an exemplary record collected for a patient with a diabetes problem;

FIG. 5 is a schematic chart showing typical contents of the report generated by the report-generating process according to one embodiment of the present invention;

FIG. 6 shows a schematic diagram that illustrates a flow of a point of care system according to a first embodiment of the present invention;

FIG. 7 shows a schematic diagram that illustrates a flow of a point of care system according to a second embodiment of the present invention;

FIGS. 8A and 8B show two different situations for the patients under the point of care health management according to the present invention;

FIG. 9 is a schematic diagram showing some known proteomic markers and physical indicators of a patient;

FIG. 10 is a schematic diagram showing a machine-learning process of modifying the analysis according to the present invention.

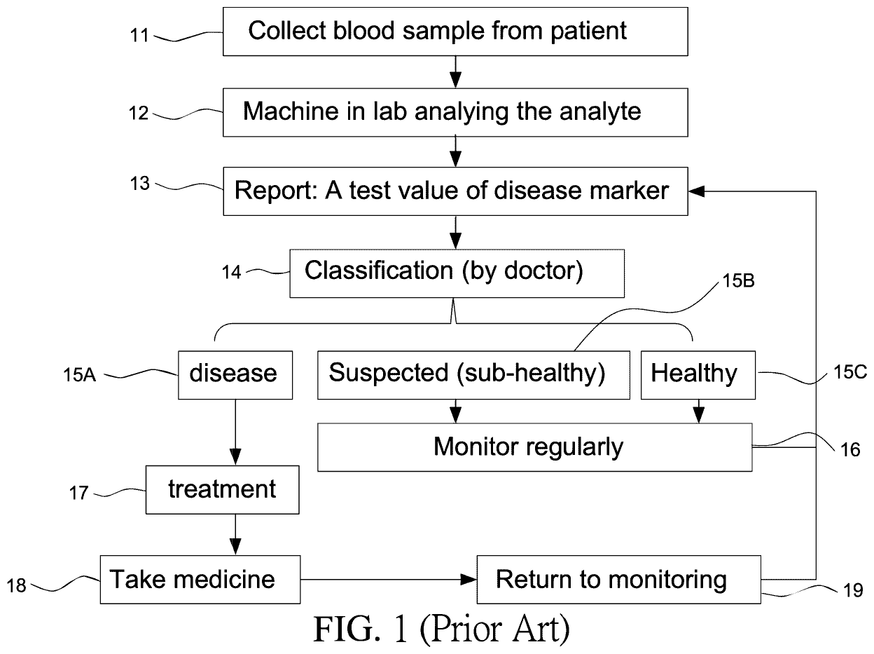

### DETAILED DESCRIPTION OF THE PREFERRED EMBODIMENTS

The present invention will now be described more specifically with reference to the following embodiments. It is to be noted that the following descriptions of the preferred embodiments of this invention are presented herein for the purpose of illustration and description only; they are not intended to be exhaustive or to be limited to the precise form disclosed.

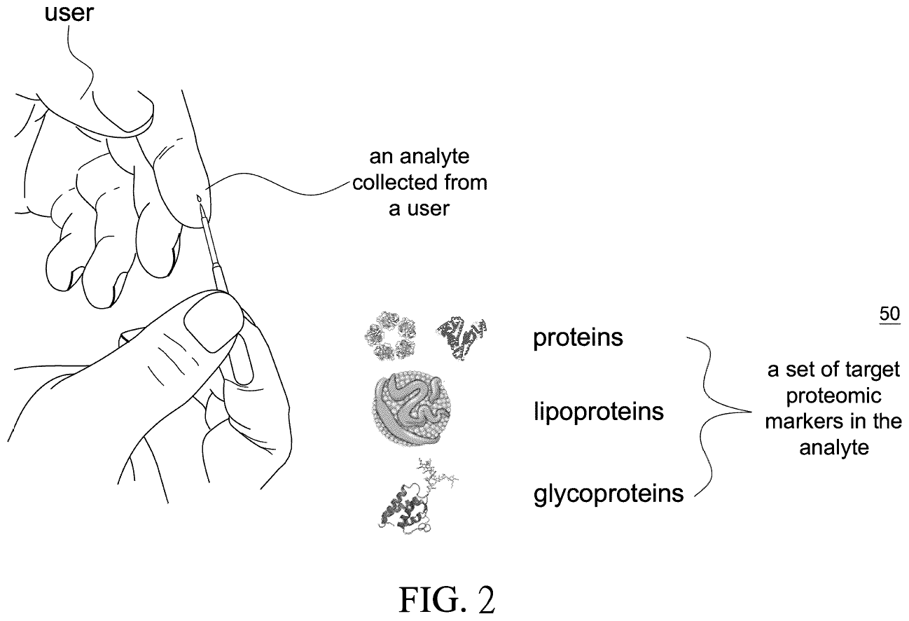

Please refer to FIG. 2, which illustrates a situation that a user, who can be a patient under the health management of a point of care system, collects an analyte 20 from his index finger by himself. The analyte can be some blood sample. For some patients suffering serious diabetes, such a simple blood test for the sugar concentration can be as many as four times per day. During the period of sepsis, measuring the inflammatory protein CRP several times a day is possible for a patient.

According to some recent researches, some proteomic markers including proteins, lipoproteins and glycoproteins can be significant indicators when determining a patient's health condition. These physiological indicators are to be combined as a whole set when employed for clinical usage. In addition, the choice of the set of target proteomic markers for a particular person or patient is highly dependent on the personal condition such as the family health history, community/environmental condition or his/her medical record in recent years. For example, an elderly person may be suffering more than one type of chronic disease, and different combinations of those proteomic markers could relate to different chronic diseases. Therefore, for a particular patient, there should be one specific set of target markers 50 determined by personnel with the expertise, such as a doctor, to be routinely monitored by the point of care system. Alternatively, the set of target markers 50 may also be determined based on the user's specific requirements if she or he feels like it.

Proteomic indicators are large-molecular materials, which can be better detected by surface acoustic waves (SAW) after reacting with corresponding reactors such as antigens or antibodies. The SAW reader can be built into a compact size for the users to operate at home. When a set of target proteomic markers for a user have been determined, the skilled person in the art can provide a corresponding set of reactors to be used for a routine inspection to the analyte collected from the user at home or a community medical center.

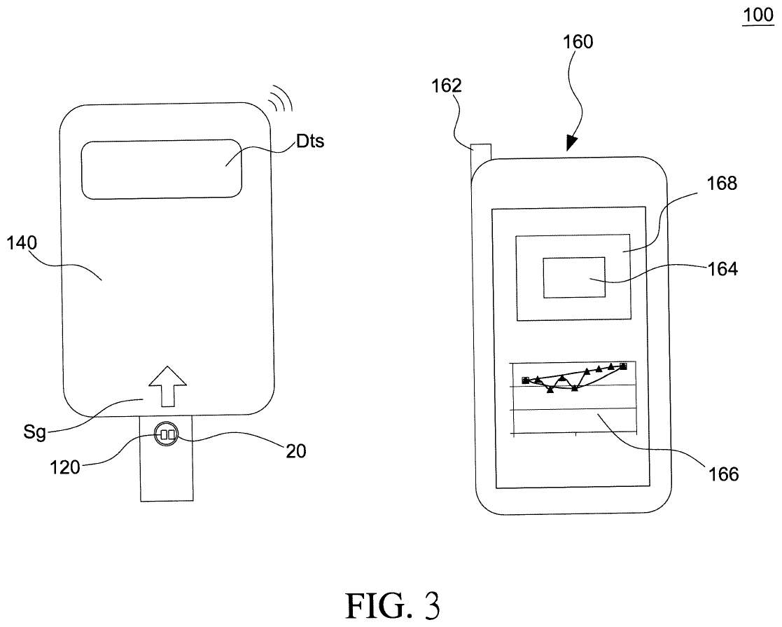

Please refer to FIG. 3, which shows a point of care system 100 according to one embodiment of the present invention. The analyte 20 collected from a user is placed into a multichannel bio-reaction device 120 such as a strip or a chip module. Depending on the set of target proteomic markers 50 predetermined as per the user's personal need, there are several corresponding reactors previously disposed in each channel of the multichannel bio-reaction device 120, and the analyte 20 will react with these reactors (not shown) for a certain period of time.

When the analyte 20 has been fully reacted with the reactors, the multichannel bio-reaction device 120 is placed into a SAW reader 140. Alternatively, the multichannel bio-reaction device 120 may be plugged into a SAW reader 140 in advance, and the analyte 20 is then placed into the multichannel bio-reaction device 120. In one embodiment, the multichannel bio-reaction device 120 is equipped with a SAW scanner providing a wide range of initial acoustic waves in terms of frequency or bandwidth. In another embodiment, the initial acoustic waves employed for detection can be provided by the SAW reader 140.

In the multichannel bio-reaction device 120, the bio-reacted analyte 20 can generate SAW signals Sg in response to the initial acoustic waves for the SAW reader 140 to detect. The SAW reader 140 can receive the SAW signals Sg and produce a plurality of time-stamped data Dts corresponding to the SAW signals Sg. The time stamp can record the date or the hour according to the time when the SAW reader 140 receives the SAW signals Sg for future analysis. The plurality of time-stamped data Dts is then transmitted out via electronic communication by the SAW reader 140.

In the point of care system 100, a user device 160, such as a cellular phone or a laptop computer, can receive the time-stamped data Dts with a communication module 162. The electronic communication can be a wireless communication such as, but not limited to, blue-tooth or any appropriate type of radio-frequency communication. In another embodiment, the electronic communication can be wired communication.

The user device 160 further includes a processor 164 configured to bear and run a report-generating process 166 which can be either a software or an application program (APP) download from the website. The user device 160 may also include a memory 168 for restoring the time-stamped data Dts received by the communication module 162 and some other information including some previously received time-stamped data or the data from difference resources. The memory 168 may also be configured to store the report-generating process 166.

The report-generating process 166 can generate a report based on the time-stamped data Dts and some historical time-stamped data relevant to the set of target proteomic markers 50, and thus an analysis can be provided based on the set of target proteomic markers 50.

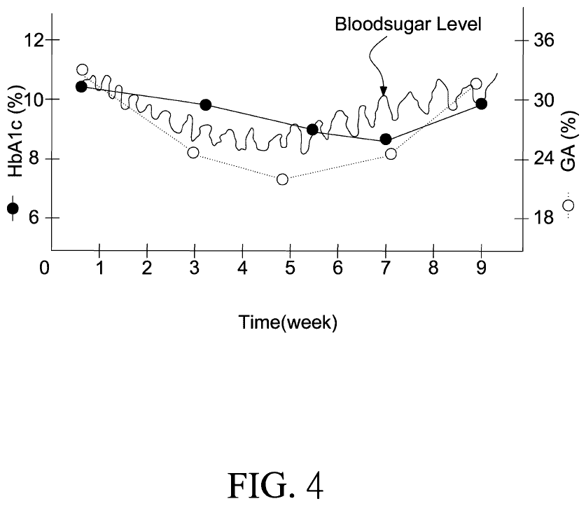

FIG. 4 shows an exemplary record collected for a patient with diabetes. For the regular health examination to the ordinary people, there are basically three indicators, namely the blood sugar, Glycated Hemolobin (HbAlc) and glycated albumin (GA), can be considered as the ones relevant to diabetes. Blood sugar concentration in a person's body can vary largely because the human body keeps absorbing sugar from food while consuming sugar when excising, while the HbAlc and GA are relatively stable and can be considered as long-term and middle-term indicators.

In FIG. 4, three indicators are shown routinely monitoring during the first period of the first three weeks when the patient is hospitalized and the second period of weeks 4-7 when the patient has recovered, and moved back home but then again goes back to the hospital for clinic checks on weeks 7 and 9.

It can be observed that, during the period of hospitalization, the blood sugar level varies while the trend thereof is moving down, the level of GA drops significantly, and the level of HbAlc is merely slightly lower than before.

It is also observed that, after the patient has moved back home, the blood sugar level still fluctuates and the trend thereof slightly bounces back, while the level of HbAlc keeps lowering until the patient's first clinical visit for physical check. It appears that the level of GA provides simple but timely information relevant to the blood sugar level. One may consider the blood sugar level, the GA level and the HbAlc as a short-term, a middle-term and a long-term indicator respectively.

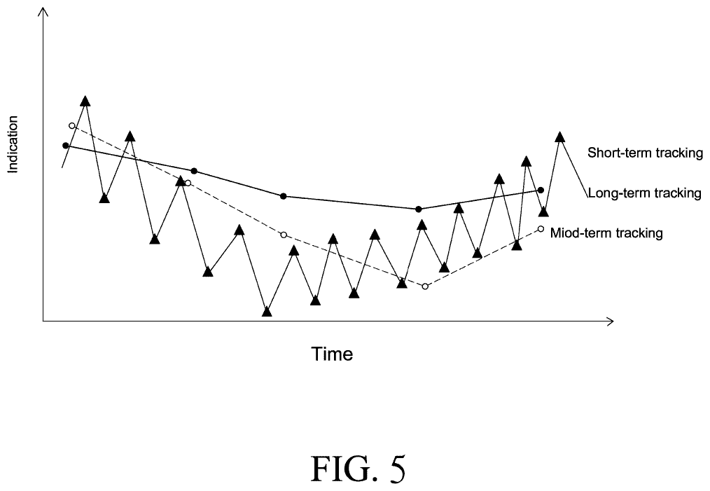

Please refer to FIG. 5, which shows some typical contents of the report generated by the report-generating process according to one embodiment of the present invention. In general, there are three typical marker indications relevant to short-term, middle-term and long-term tracking in one report. It is appreciated by the skilled person in the art that the chart in FIG. 4 shows markers related to diabetes. In other embodiments, the target proteomic markers can relate to cardiovascular diseases which are popular or the elderly people. In some other embodiments, the set of target proteomic markers can relate to several chronic diseases for a general purpose of use. The different markers for different types of tracking in terms of time frequency should be considered altogether for determining the health condition of the patients, according to the practice of experienced medical personnel. Therefore, the analysis based on a set of markers is rather valuable for the health care system.

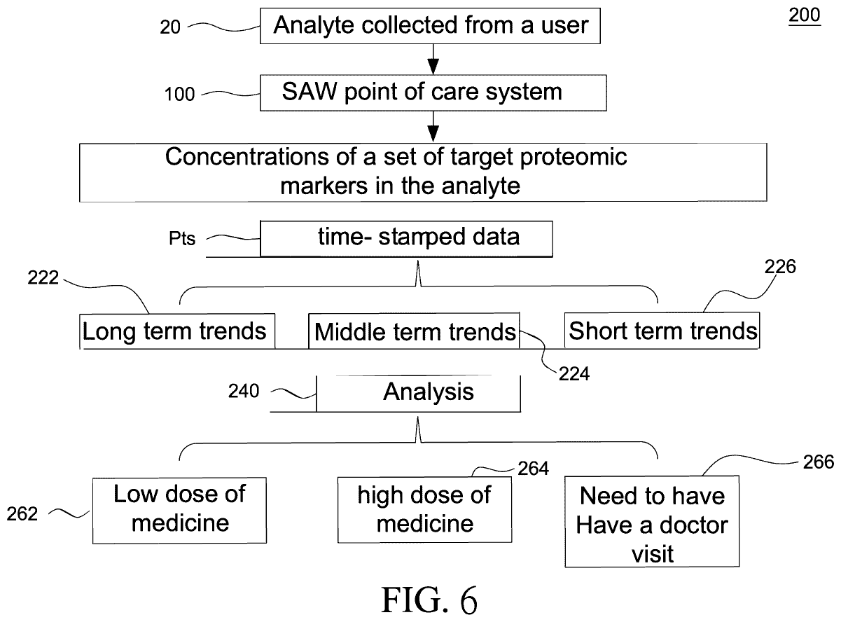

Please refer to FIG. 6, which illustrates a flow 200 applying the point of care system 100 according to a first embodiment of the present invention. The analyte 20 such as a blood sample collected from a user can be checked by the devices in the point of care system 100, based on a set of target proteomic markers previously determined by the user or the doctor, the concentration data of the markers can be verified by the devices, and the time-stamped data Pts is available for analysis.

There are three types of trends that can be determined based on the time-stamped data Pts, namely long term trends 222, middle term trends 224 and short term trends 226. All the different types of trends are put into an analysis step 240 for a professional evaluation, and the result will be one of a low dose of medicine 262, a high dose of medicine 264 and a need to have a doctor to visit 266, depending on the patient's health situation.

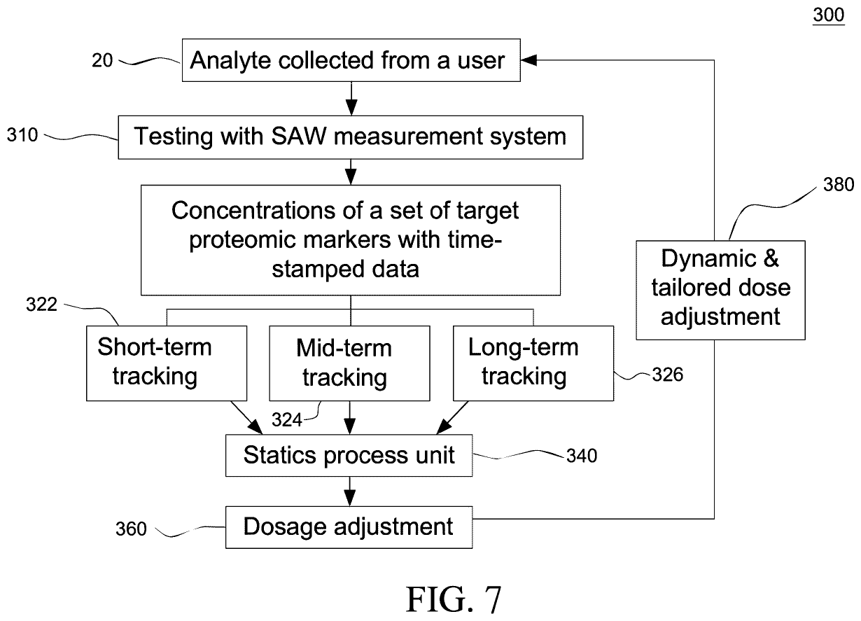

Please refer to FIG. 7, which illustrates a flow 300 according to a second embodiment of the present invention. The analyte 20 such as a blood sample collected from a user can be tested by the SAW measurement device in the point of care system 100, based on a set of target proteomic markers previously determined by the user or the doctor, the concentration data of the markers can be verified by the devices, and the time-stamped data Pts is available for analysis. In this flow 300, the user device 160 can be alternative, because the calculations of the concentration data can be handled by either a local device or a remote device.

Three types of tracking can be made, based on the time-stamped data Pts, namely short-term tracking 322, middle-term tracking 324 and long-term tracking 326. All the different types of trends are put into a statistical process unit 340 for analysis, and the result will be subject to a dosage adjustment 360 and a dynamic and tailored dose adjustment 380 in the long run. This becomes a continuous loop for the point of care system. Notably, the point of care system 10 can be used to monitor and regulate a blood sugar and lipid levels, and also be used to adjust medication usage to minimize side effects.

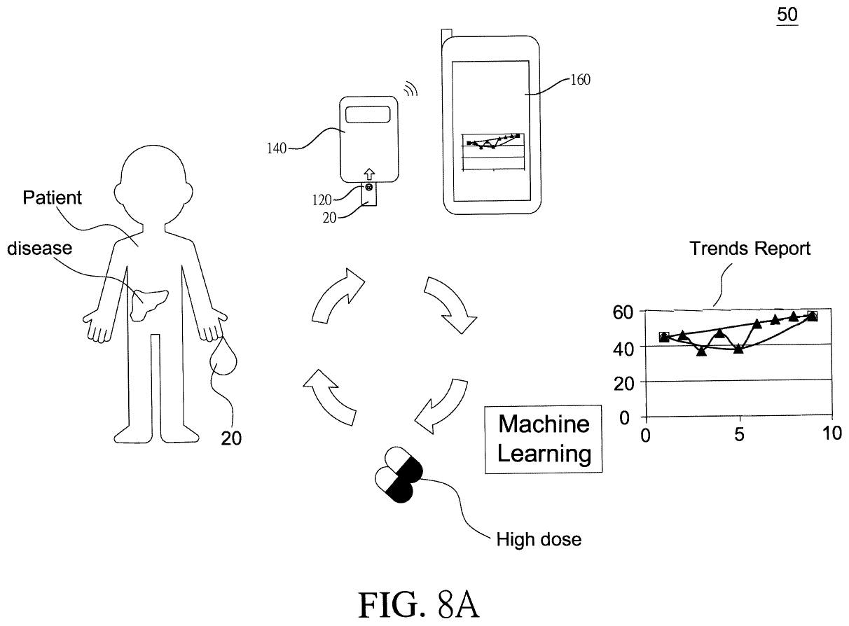

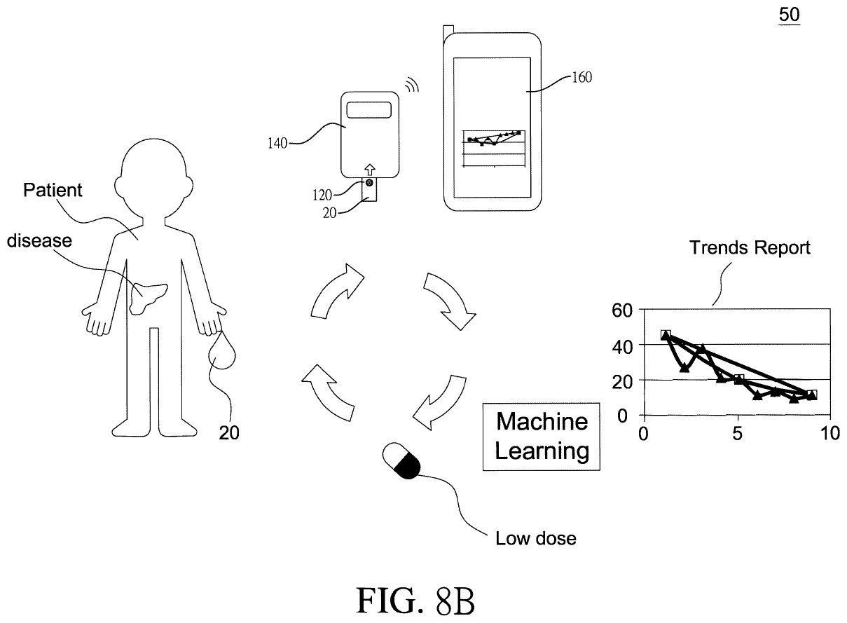

FIGS. 8A and 8B show two different situations for the patients under the point of care health management according to the present invention. Notably, the patient is suffering chronic diseases but without a need to go to the hospital, which conserves the medical resources.

In FIG. 8A, it is observed that the indicator trend in the trends report moves slightly upwards, and thus a high dosage for the medicine is required for the patient to maintain his/her health condition. A machine learning process can be applicable to further analyze the collected time-stamped data and figure out a more accurate dosage of the medicine to be taken by the patient.

In FIG. 8B, it is observed that the indicator trend in the trends report moves slightly downwards, and thus a low dosage for the medicine is suggested for the patient to maintain his/her health condition. Likewise, the machine learning process can also be applicable to further analyze the collected time-stamped data and figure out a more accurate dosage of the medicine to be taken by the patient. The machine learning process can be based on the collected time-stamped data from the point of care system 100 and any other information from remote medical research centers or any relevant inspection data collected from the same community.

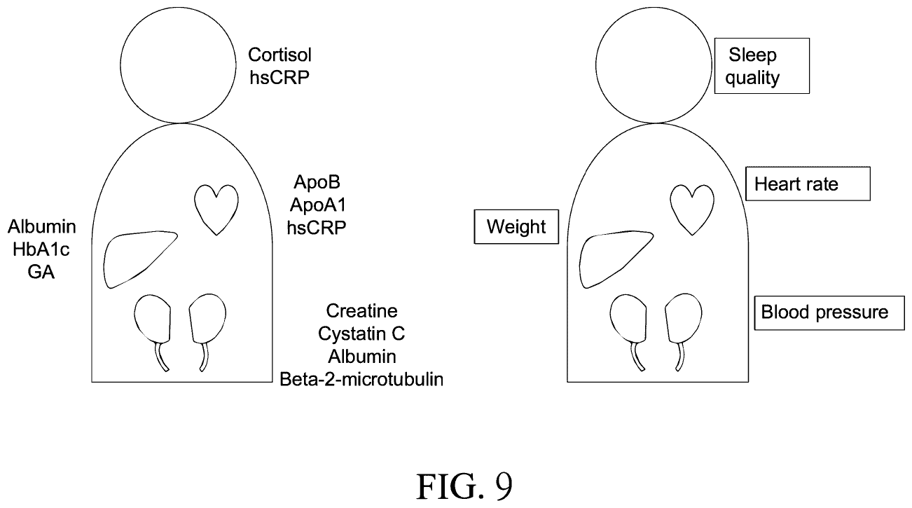

FIG. 9 provides some ideal of combining the known proteomic markers and some physical indicators of one patient to specify a set of comprehensive indicators for the analysis.

It is appreciated by the skilled person in the art that, the combination of these selected indicators can be cross-relevant to several chronic diseases, and the actual relationship between the two can be highly dependent on the personal condition of each patient. Therefore, historical data is still valuable for the analysis in the future.

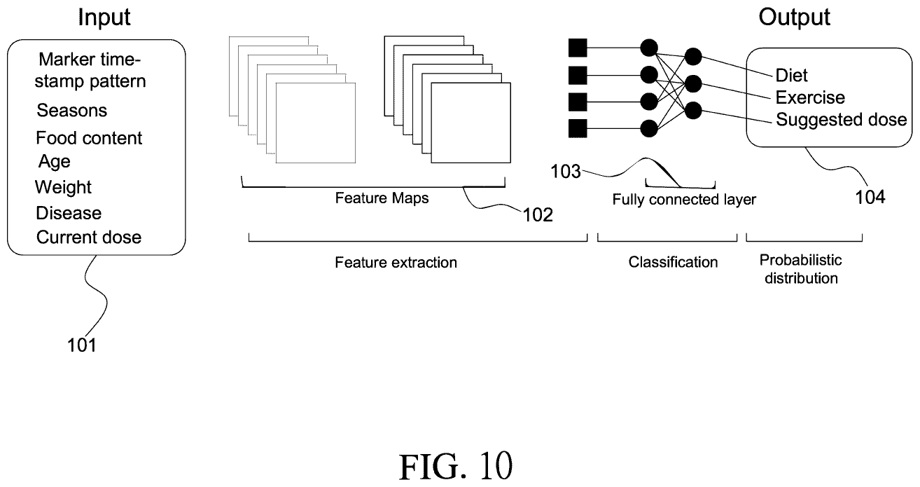

FIG. 10 is a schematic diagram showing a machine-learning process of modifying the analysis according to the present invention. In a typical machine-learning process, there should be a set of input information collected from the feature maps for the feature extraction. A classification step following the feature extraction is performed, and a collection between the classified indicators and the output such as diet plus medical treatment and the exercising plan is then determined. The machine-learning process can also help to modify the contents of the set of target proteomic markers for a specific person.

While the invention has been described in terms of what is presently considered to be the most practical and preferred Embodiments, it is to be understood that the invention need not be limited to the disclosed Embodiments. On the contrary, it is intended to cover various modifications and similar arrangements included within the spirit and scope of the appended claims, which are to be accorded with the broadest interpretation so as to encompass all such modifications and similar structures.

1. A point of care system comprising:
a multichannel bio-reaction device configured to receive an analyte collected from a user and generate a plurality of surface acoustic wave (SAW) signals each of which corresponds to at least one of a set of target proteomic markers in the analyte;
an SAW signal reader configured to receive the SAW signals and produce a plurality of time-stamped data corresponding to the SAW signals; and
a communication module configured to receive the plurality of time-stamped data and transmit the received plurality of time-stamped data to a processor, wherein the processor is configured to run a report-generating process for generating a report including an analysis to the set of target proteomic markers.

2. The point of care system as claimed in claim 1, further comprising a memory device connecting to the processor and configured to store the plurality of time-stamped data and historical time-stamped data related to the set of target proteomic markers.

3. The point of care system as claimed in claim 2, wherein the analysis is based on the plurality of time-stamped data and the historical time-stamped data.

4. The point of care system as claimed in claim 1, wherein the set of target proteomic markers are predetermined based on the user's specific requirements.

5. The point of care system as claimed in claim 1, wherein the set of target proteomic markers includes at least a lipoprotein and a glycoprotein.

6. The point of care system as claimed in claim 1, wherein the set of target proteomic markers are related to at least a cardiovascular disease and a renal disease.

7. The point of care system as claimed in claim 1, wherein the report further includes an additional analysis to one of the set of target proteomic markers related to the user's community members.

8. The point of care system as claimed in claim 1, wherein the report includes a short term trend, a middle term trend and a long term trend.

9. The point of care system as claimed in claim 1, wherein the user device is connected to a statistic process unit configured to perform a feature extraction and a feature classification based on a set of input data including the plurality of time-stamped data, the historical time-stamped data and fundamental information related to the user.

10. The point of care system as claimed in claim 9, wherein the feature extraction and the feature classification are performed by means of machine learning.

11. A method of providing a report for a user adopting a point of care system including a multichannel bio-reaction device, a surface acoustic wave (SAW) reader, a user device, a memory device and a report-generating process running on a processor, the method comprising steps of:
the multichannel bio-reaction device receiving an analyte collected from the user, and generating SAW signals based on a set of target proteomic markers in the analyte;
the SAW reader receiving the SAW signals, and producing a plurality of time-stamped data corresponding to the SAW signals;
the user device receiving the plurality of time-sampled data, and transmitting the received data to the processor; and
the report-generating process generating a report including an analysis to the set of target proteomic markers related to the user.

12. The method as claimed in claim 11, wherein the memory device stores the plurality of time-stamped data and historical time-stamped data related to the set of target proteomic markers.

13. The method as claimed in claim 12, wherein the analysis is done based on the plurality of time-stamped data and the historical time-stamped data.

14. The method as claimed in claim 11, wherein the set of target proteomic markers are predetermined based on the user's specific requirements.

15. The method as claimed in claim 11, wherein the set of target proteomic markers includes a lipoprotein and a glycoprotein.

16. The method as claimed in claim 11, wherein the set of target proteomic markers are related to a cardiovascular disease.

17. The method as claimed in claim 11, wherein the report further includes an additional analysis done to one of the set of target proteomic markers related to the user's community members.

18. The method as claimed in claim 11, wherein the report includes a short term trend, a middle term trend and a long term trend.

19. The method as claimed in claim 11, wherein the user device is connected to a statistic process unit, and the method further comprises a sub-step of having the statistic process unit perform a feature extraction and a feature classification based on a set of input data including the plurality of time-stamped data, the historical time-stamped data and fundamental information related to the user, and wherein the point of care system is used to monitor and regulate a blood sugar and lipid levels, and also used to adjust medication usage to minimize side effects.

20. A point of care system comprising:
a multichannel bio-reaction device configured to receive an analyte collected from a user;
a signal generator, in response to a receipt of the analyte, generating a plurality of surface acoustic wave (SAW) signals each of which corresponds to at least one of a set of target proteomic markers in the analyte; and
a reporter generating a report including an analysis to the set of target proteomic markers.
